(19) United States
(12) Patent Application Publication
(10) Pub. No.: US 2009/0162368 A1
(43) Pub. Date: Jun. 25, 2009

(54) MECHANOSENSITIVE MAMMALIAN POTASSIUM CHANNEL ACTIVATABLE BY POLYUNSATURATED FATTY ACIDS

(75) Inventors: Eric Honore, Juan les Pins (FR); Michel Fink, Bagnols sur Ceze (FR); Michel Lazdunski, Nice (FR); Florian Lesage, Valbonne (FR); Fabrice Duprat, Vlougins (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Centre National de la Recherche Scientifique-CNRS, corporation of France, Paris Cedex (FR)

(21) Appl. No.: 12/329,381

(22) Filed: Dec. 5, 2008

Related U.S. Application Data
(63) Continuation-in-part of application No. 11/224,260, filed on Sep. 12, 2005, now Pat. No. 7,468,422.
Application No. 11/224,260 is a division of application No. 09/655,272, filed on Sep. 5, 2000, now Pat. No. 6,942,979.
Application No. 09/655,272 is a continuation of application No. PCT/FR99/00404, filed on Feb. 23, 1999.

(30) Foreign Application Priority Data
Mar. 5, 1998 (FR) 98/02725

(51) Int. Cl. A61K 39/395; C07K 14/00; C07K 16/18; C12N 15/11; C12N 15/00; C12P 21/02; C12N 15/87; C12N 5/06; C12Q 1/02; A61K 38/16; A61K 31/7088

(52) U.S. Cl. 424/139.1; 530/350; 530/387.9; 536/23.5; 435/320.1; 435/69.1; 435/455; 435/365; 435/29; 514/12; 514/44

(57) ABSTRACT

A purified protein comprising a mechanosensitive potassium channel activated by at least one polyunsaturated fatty acid and riluzole and the use of said channels in drug screening.

### RELATED APPLICATION

This application is a continuation-in-part of application Ser. No. 11/224,260, filed Sep. 12, 2005, which is a divisional of application Ser. No. 09/655,272, filed Sep. 5, 2000, now U.S. Pat. No. 6,942,979, which is a continuation of International Application No. PCT/FR99/040404, with, an international filing date of Feb. 23, 1999, which is based on French Patent Application No. 98/02725, filed Mar. 5, 1998, incorporated herein by reference.

### TECHNICAL FIELD

This disclosure concerns a new class of mechanosensitive potassium channels activated by polyunsaturated, fatty acids. The disclosure is based on the discovery of a new mechanosensitive potassium channel, sometimes hereinafter referred to as “TRAAK” as an abbreviation for TWIK-Related AA-ACTIVATED K⁺ channel, which is activated by polyunsaturated fatty acids as well as by the neuroprotective agent riluzole. The properties of the channels of the TRAAK family as well as their tissue distribution give these channels a primordial role in the transport of potassium in a large number of cell types.

### BACKGROUND

Potassium channels are ubiquitous proteins and their exceptional functional diversity makes them ideal candidates for a large number of biological processes. They intervene notably in the regulation of neuronal and muscular excitability, cardiac rhythm and hormone secretion. Three structural types of potassium channels have been described in mammals. The first is the Shaker type which is composed of subunits that have, six transmembranal segments and one P domain which is implicated in the formation of the ionic pore. The second is the IRK type which has two transmembranal segments and one P domain. The third has been described more recently and corresponds to the TWIK type which has four transmembranal segments and two P domains. Three channels of this type have been identified: TWIK-1 (Fink, M. et al. EMBO J. 15, 6854-6862 [1996]; Lesage, F. et al. EMBO J. 15, 1004-1011 [1996]), TREK-1 and TASK.

(Duprat, F: et al. EMBO J 16, 5464-5471 [1997]). In addition to a conserved general structure, they have primary sequences exhibiting little similarity since they present between 20 and 25% amino acid identity.

### SUMMARY

We accordingly provide, among other things, a purified protein, antibodies, nucleic acids, vectors and various methods as follows:
a purified protein comprising a mechanosensitive potassium channel activated by at least one polyunsaturated fatty acid and riluzole;
a purified nucleic acid molecule comprising a nucleic acid sequence encoding the protein;
a vector comprising, the purified nucleic acid molecule operably linked to regulatory sequences;
a method for producing the purified protein comprising:
a) transferring the nucleic acid molecule into a cellular host;
b) culturing, the host under suitable conditions to produce a protein comprising a potassium channel; and
c) isolating the protein of step (b);
a method for expressing the potassium channel comprising:
a) transferring the purified nucleic acid molecule into a cellular host; and
b) culturing the host under suitable conditions for expressing the potassium channel;
a cellular host produced by the method;
a method for screening substances capable of modulating the activity of the purified protein comprising:
a) reacting varying amounts of the substance to be screened with the cellular host; and
b) measuring the effect of the substance to be screened on a potassium channel expressed by the cellular host;
a method for preventing or treating heart disease in mammals which comprises administering a therapeutically effective amount of a pharmaceutical composition comprising a therapeutically effective amount of a substance capable of modulating the activity of the purified protein;
a method for preventing or treating central nervous system disease in mammals which comprises administering a therapeutically effective amount of a pharmaceutical composition comprising a therapeutically effective amount of a substance capable of modulating the activity of the purified protein; and
a pharmaceutical composition comprising a therapeutically effective amount of the purified protein and a pharmaceutically acceptable carrier.

### BRIEF DESCRIPTION OF THE DRAWINGS

Other advantages and characteristics will become apparent upon reading the text and examples below which explain the identification and characterization of these mechanosensitive potassium channels which are activated by fatty acids. These examples will refer to the attached sequences and drawings in which:

FIG. 1, which contains SEQ ID NO: 1, represents the nucleotide sequence of the cDNA of TRAAK and the amino acid sequence (SEQ ID NO: 2) of the coding sequence.

FIG. 2 represents alignment of the sequences of TWIK-1 (SEQ ID NO; 5), TREK-1 (SEQ ID NO: 4), TASK (SEQ ID NO: 6) and TRAAK (SEQ ID NO: 2) which are four channels, of the TWIK type presently cloned in mammals as well as the deduced dendrogram of this alignment;

FIG. 3 represents the RT-PCR analysis of the distribution of TREK-1 and TRAAK in the tissues of the adult mouse.

FIG. 4 shows the electrophysiological properties of the TRAAK currents recorded using the imposed voltage technique on Xenopus oocytes that had received an injection of TRAAK cRNA (a, b, c) and on COS cells transfected with, a vector expressing TRAAK (d, e).

FIGS. 5a and b are graphs showing, the effect of the osmolarity of the external medium on oocytes that received an injection of TREK-1 or TASK cRNA.

FIGS. 6a-h are graphs showing that TREK-1 is a mechanosensitive potassium channel in the transfected COS cells.

FIG. 7 shows, the activation of TRAAK by stretching the cellular membrane in the transfected COS cells.

FIGS. 8a-f are graphs showing the activation of TREK-1 by arachidonic acid in the transfected COS cells.

FIGS. 9a-e are graphs showing the effect of arachidonic acid and other fatty acids on the TRAAK channel expressed in the transfected COS cells.

FIGS. 10a and b are graphs showing the effect of riluzole on the TREK-1 and TRAAK designated TREK-2 currents.

### DETAILED DESCRIPTION

We discovered and cloned a new channel designated TRAAK, which is a member of the TWIK channel family. The gene coding this channel is most particularly homologous at the level of its amino acid sequence with the TREK-1 channel with which it exhibits 38% amino acid identity. The disclosure is also based on the unique electrophysiological properties of the TREK-1 and TRAAK channels. In fact, both of these channels produce potassium-selective currents which are activated by a tension applied to the cell membrane, which channels are referred to as mechanosensitive, or by the application of polyunsaturated fatty acids, especially arachidonic acid which is an essential messenger of intercellular and intracellular communication and an important modulator of neuronal excitability (Ordway, R. W., Singer, J. J. and Walsh, J. V. 14, 96-100 [1991]; Bliss, T. V. P. and Collingridge; G. L. Nature 31-39 [1993]; Piomelli, D. Curr. Opin. Cell Biol. 5, 274-280 [1993]; Meves, H. Prog. Neurobiol. 43, 175-186 [1994]; Piomelli, D. Crit. Rev; Neurobiol. 8,65-83 [1994]. These channels, are also opened by riluzole which is a neuroprotective agent (Malgouris, C. et al. J. Neurosci. 9, 3720-3727-[1989]; Pratt, J. et al. Neuroscience. Lett. 140, 225-230 [1992]) used clinically to prolong the lives of patients with amyotrophic lateral sclerosis.

The discovery of this new class of potassium channels and the heterologous expression of these channels provides us notably with new research tools for screening drugs that are capable of modulating the activity of the potassium channels and thus of preventing or treating diseases implicating these channels such as epilepsy, cardiac pathologies (arrhythmias) and Vascular diseases, neurodegenerative diseases, especially those associated with ischemia and anoxia, the endocrine diseases associated with defective hormone secretion and muscle diseases.

Thus, we provide a purified protein constituting a mechanosensitive potassium channel activated by polyunsaturated fatty acids, especially arachidonic acid, and by riluzole. More specifically, we provide protein constituting the TRAAK channel, the amino acid sequence of which is represented in the attached sequence list as SEQ ID NO: 2 or a functionally equivalent derivative of this protein.

Such derivatives include those with a sequence comprising a modification and/or a suppression and/or an addition of one or more amino acid residues, as long as this modification and/or suppression and/or addition does not modify the properties of the TRAAK channel. Such derivatives cant be analyzed by the expert in the field using the techniques described in the examples presented below which enable demonstration of the biophysical and pharmacological properties of the TRAAK channel. More specifically, such a derivative is the TREK-1 channel the amino acid sequence of which is represented in the attached sequence list as SEQ ID NO: 4.

Polyclonal or monoclonal antibodies directed against at least one protein constituting an ionic channel can be prepared by the classic methods described in the literature. These antibodies are useful for detecting the presence of the ionic channels in various human and animal tissues; however, because of their specificity, they can also find therapeutic applications for the in vivo inhibition or activation of a TRAAK channel and/or its derivatives.

We also provide a purified nucleic acid molecule comprising or constituted by a nucleic sequence coding for a protein constituting a mechanosensitive potassium channel activated by polyunsaturated fatty acids, especially arachidonic acid, and by riluzole. More specifically, we provide a nucleic acid molecule comprising at least one sequence coding for the protein constituting the TRAAK channel, the amino acid sequence of which is represented in the attached sequence list as SEQ ID NO: 2 or for a functionally equivalent derivative of this protein. A DNA molecule comprising the sequence coding for the TRAAK protein is represented in the attached sequence list as SEQ ID NO: 1 or its complementary sequence. More specifically, such a nucleic acid sequence comprises the sequence between nucleotides 284 and 1477 of SEQ ID NO: 1 or its complementary sequence.

Another nucleic acid sequence comprises at least one sequence coding for the protein constituting the TREK-1 channel which has the amino acid sequence represented in the attached sequence list as: SEQ ID NO: 4 or for a functionally equivalent derivative of this protein. A DNA molecule comprising the sequence coding for the TREK-1 protein is represented in the attached sequence list as SEQ ID NO: 3 or its complementary sequence. More specifically, such an amino acid sequence comprises the sequence between nucleotides 484 and 1596 of SEQ ID NO: 3.

We also provide a vector comprising at least one of the preceding nucleic acid molecules, advantageously associated with suitable control sequences, as well as a process for the production or expression in a cellular host of a protein constituting an ionic channel. The preparation of these vectors as well as the production or expression in a host of the channels can be implemented by molecular biology and genetic engineering techniques which are well known to the expert in the field.

As an example, a process for the production of a protein constituting a cationic channel comprises:
transferring a nucleic acid molecule or a vector containing the molecule into a cellular host,
culturing the cellular host under conditions enabling production of the protein constituting the potassium channel,
isolating by any suitable means the proteins constituting the potassium channels.

As an example, a process for the expression of an ionic channel comprises:
transferring a nucleic acid molecule or a vector containing the molecule into a cellular host,
culturing the cellular host under condition is enabling expression of the potassium channels.

The cellular host employed in the preceding processes can be selected from among the prokaryotes or the eukaryotes and especially from among the bacteria, yeasts, and mammal, plant or insect cells.

The vector employed is selected on the basis of the host into which it will be transferred; all vectors such as plasmids can be employed.

Thus, the disclosure also pertains to the cellular hosts and more specifically the transformed cells expressing the potassium channels exhibiting the properties and structure of the type of TRAAK channel cells obtained in accordance with the preceding processes. These cells are useful for screening substances capable of modulating the TRAAK channel currents. This screening is implemented by bringing into contact, variable quantities of a substance to be tested with cells expressing the channels, then measuring by any suitable means the possible effects of the substance on the potassium currents of the channels. Electophysiological techniques also make these studies possible and are also the object when employed with TRAAK channels or their derivatives. This screening process makes it possible to identify drugs that can modulate the activity of the potassium channels, and thus might be able to prevent or treat the diseases in which these channels are implicated. These substances and their use as drugs, isolated and detected by means of the above process, are also part of the disclosure.

More specifically, we provide a chemical or biological substance capable of modifying the currents of a potassium channel for the preparation of a drug that is useful in the prevention or treatment of diseases of the heart or nervous system in human or animal subjects, such as cardiac pathologies (arrhythmias) and vascular diseases, neurodegenerative diseases, especially those associated with ischemia and anoxia, endocrine diseases associated with defective hormone secretion and muscle diseases.

A nucleic acid molecule coding for a protein constituting a TRAAK channel or a derivative thereof, or a vector comprising this nucleic acid molecule or a cell expressing TRAAK channels are also useful for the preparation of transgenic animals. These can be animals that overexpress the channels, but more especially knock-out animals, e.g., animals presenting a deficiency in these channels; these transgenic animals are prepared by methods which are known to the expert in the field, and allow preparation of live models for studying the animal pathologies associated with the TRAAK channels.

These transgenic animals as well as the previously described cellular hosts are useful as models for studying the pathologies associated with these mechanosensitive, potassium channels which are activated by polyunsaturated fatty acids either because they overexpress the potassium channels of the TRAAK channel type or because they present a deficiency in these potassium channels.

In addition, a protein constituting a neuronal ionic TRAAK channel can also be useful for the manufacture of drugs intended to treat or prevent the diseases in which these channels are implicated. The disclosure thus also pertains to the pharmaceutical compositions comprising as active principle at least one of these proteins possibly combined with a physiologically acceptable vehicle.

In fact, the nucleic acid molecules or the cells transformed by the molecules are suitable for use in gene therapy strategies to compensate for a TRAAK channel deficiency at the level of one or more tissues of a patient. The disclosure thus also pertains to a drug comprising the nucleic acid molecules or cells transformed by the molecules for the treatment of diseases in which the TRAAK channels or their derivatives are implicated.

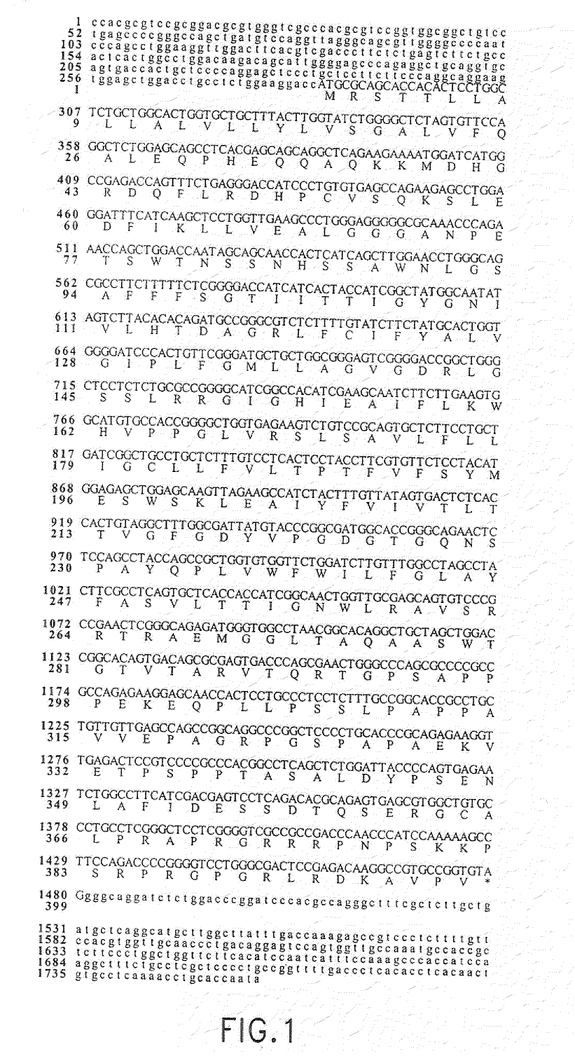

FIG. 1, which contains SEQ ID NO: 1, represents the nucleotide sequence of the cDNA of TRAAK and the corresponding amino acid sequence SEQ ID NO: 2.

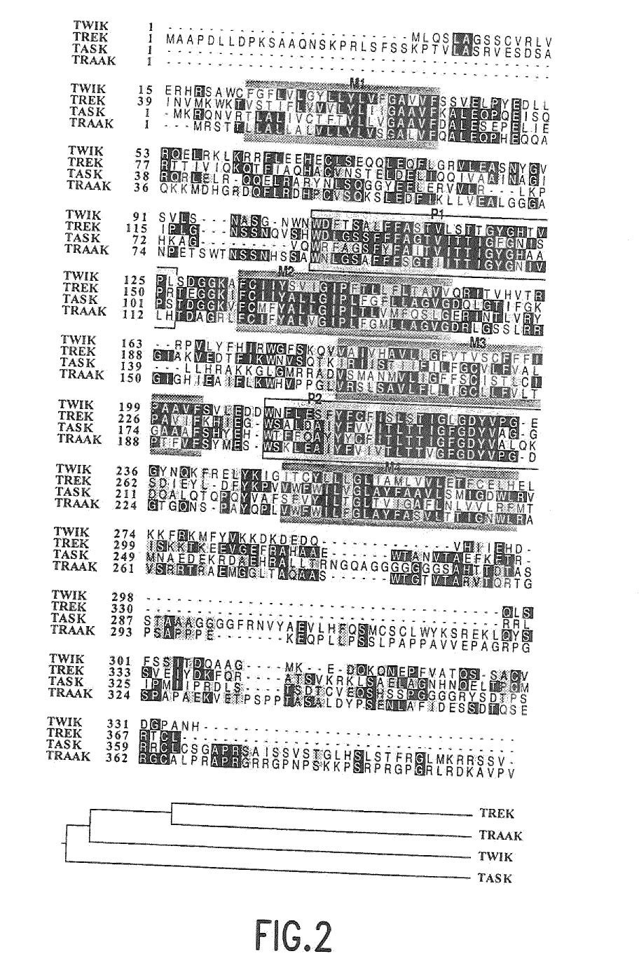

FIG. 2 represents alignment of the sequences of TWIK-1 (SEQ ID NO: 5), TREK-1 (SEQ ID NO: 4), TASK (SEQ ID NO: 6) and TRAAK (SEQ ID NO: 2), which are four channels of the TWIK type presently cloned in mammals as well as the deduced dendrogram of this alignment. Identical residues are represented on a black background and the conserved residues are represented on a gray background.

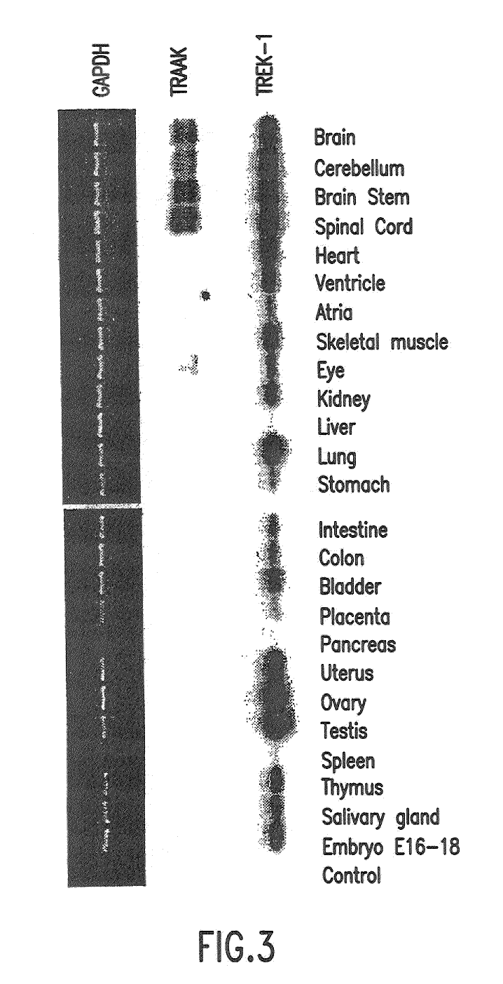

FIG. 3 represents the RT-PCR analysis of the distribution of TREK-1 and TRAAK in the tissues of the adult mouse. Fragments of the transcripts coding for TREK-1 and TRAAK were amplified by PCR using specific oligonucleotides, transferred onto a nylon membrane then labeled with oligonucleotides internally marked with phosphorus 32.

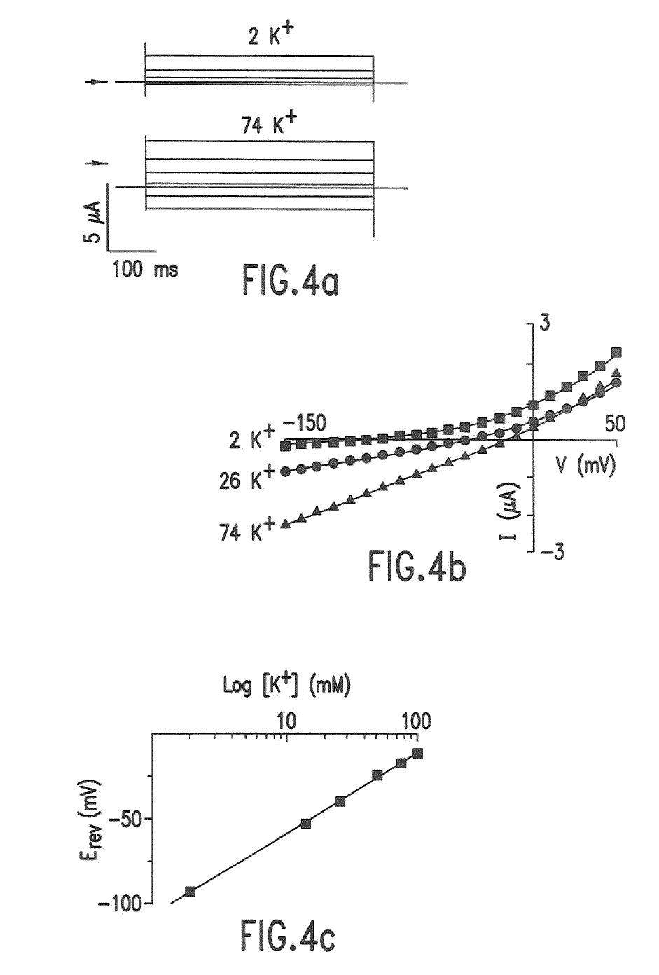

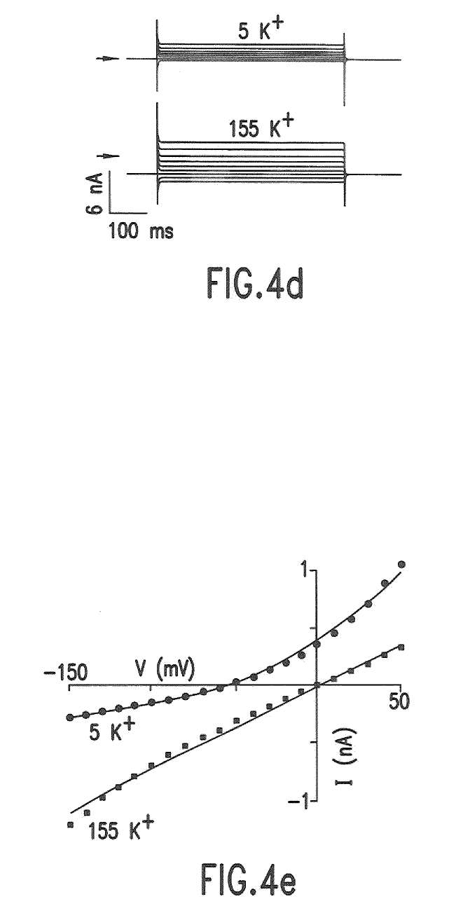

FIG. 4 shows the electrophysiological, properties of the TRAAK currents recorded using the imposed voltage technique on Xenopus oocytes that had received an injection of TRAAK cRNA (a, b, c) and on COS cells transfected with a vector expressing TRAAK (d, e, f). In (a): the oocytes were maintained at a potential of −80 mV then the currents were recorded following potential jumps from −150 to +50 mV by increments of 20 mV. The recordings were performed in an external medium containing a K⁺ concentration of 2 mM or 74 mM. In (b): current-potential relation was according to the same experimental set-up as in (a). In (c): potential reversal (Eᵣₑᵥ) of the TRAAK currents were a function of the external K⁺ concentration. In (d): currents recorded on COS cells transfected by TRAAK according to the same protocol as in (a). In (e): current-potential relation was according to the same experimental set-up as in (d).

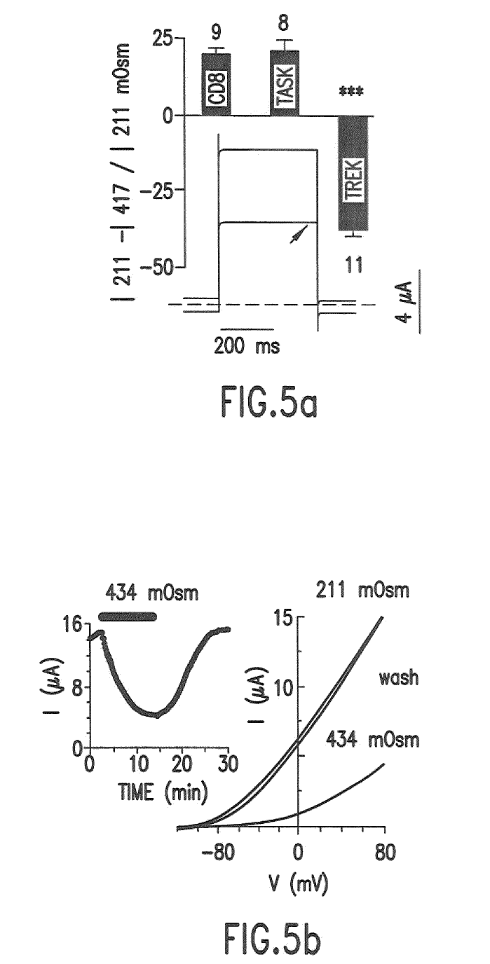

FIG. 5 shows, the effect of the osmolarity of the external medium on oocytes that received an injection of TREK-1 or TASK cRNA. In FIG. 5a: comparison of the effects of the application of a hypertonic solution (417 mOsm, by addition of mannitol) on control oocytes (CD8) and on oocytes expressing TASK or TREK-1 are shown. The currents were measured after a potential jump from −80 to +80 mV. The inset shows the TREK-1 current before and after (indicated by an arrow) the application of the hypertonic solution. In FIG. 5b: reversible effect of a hypertonic solution (434 in Osm, by addition of sucrose) on the current-potential relations deduced from the potential ramps which lasted 600 msec is shown. The inset shows the kinetics of the effect produced by the hypertonic solution. The currents were measured at 80 mV.

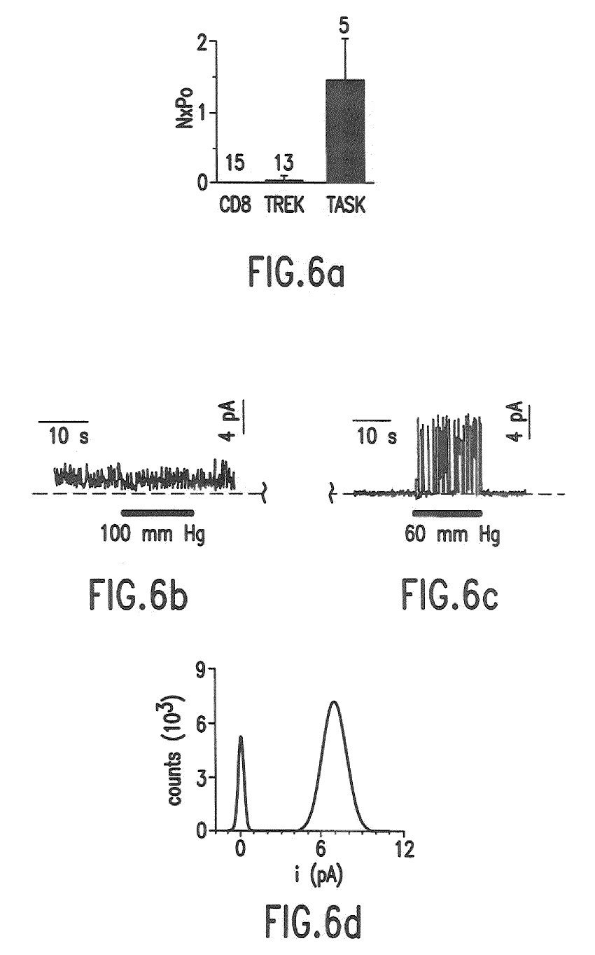

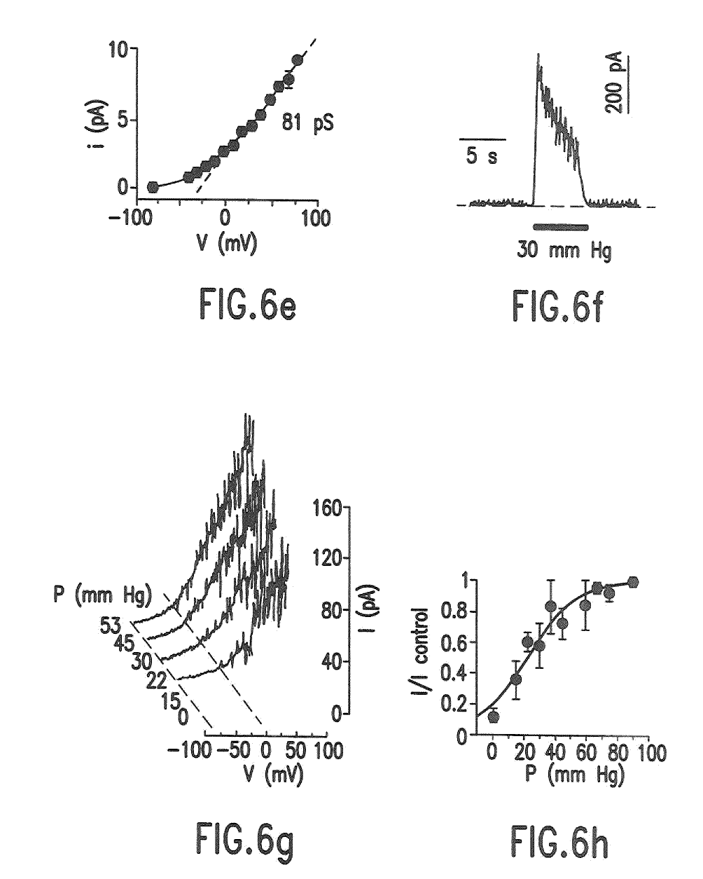

FIG. 6 shows that TREK-1 is a mechanosensitive potassium channel in the transfected COS cells. In FIG. 6a: channel activities (N*Po) in the membrane patches were maintained at 0 mV and obtained in the attached cell configuration from control cells (CD8) or from cells transfected by TREK-1 and TASK. In FIG. 6b: stretching the membrane had no effect on the activity of the TASK channel (attached cell configuration). The Piatch was maintained at 50 mV. In FIG. 6c: the TREK-1 channels were silent at rest and opened upon tension of the membrane. The patch was maintained at +50 mV. In FIG. 6d: the histogram shows the amplitude of the channel activity generated by the membrane tension and illustrated in FIG. 6f. In FIG. 6e: current-potential relation in a single TREK-1 channel (n=6) is seen. The conductance of 81 pS, was calculated between 0 and 80 mV. In FIG. 6f: activation of TREK-1 by stretching the membrane (30 mmHg) in the inside-out configuration is shown. The maintenance potential was 100 mV. In FIG. 6g: effects produced by higher and higher tensions (5 seconds duration) on the current-potential relation of a patch expressing TREK-1 are shown. In FIG. 6h: dose-effect curve of the activation, of TREK-1 by the tension (n=6) is seen. The curve was traced by following the experimental points according to the Boltzmann relation.

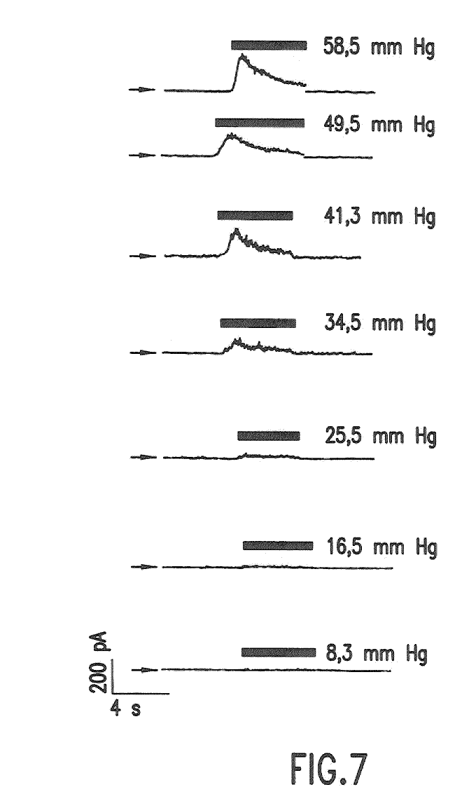

FIG. 7 shows the activation of TRAAK by stretching the cellular membrane in the transfected COS cells. The current was recorded at 0 mV in the inside-out configuration. The depressions applied via the recording pipette are indicated to the right of the tracings.

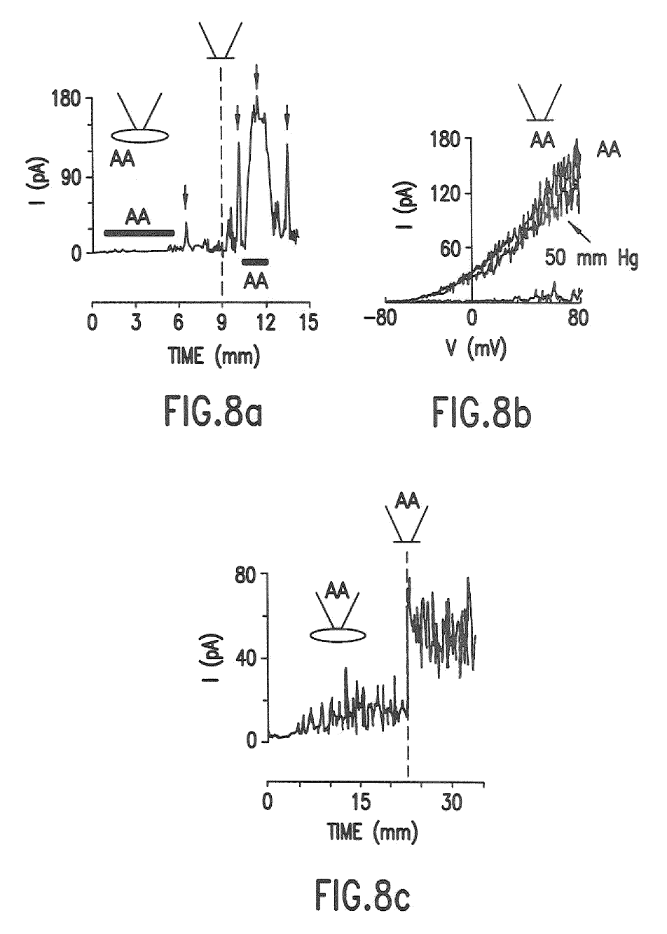

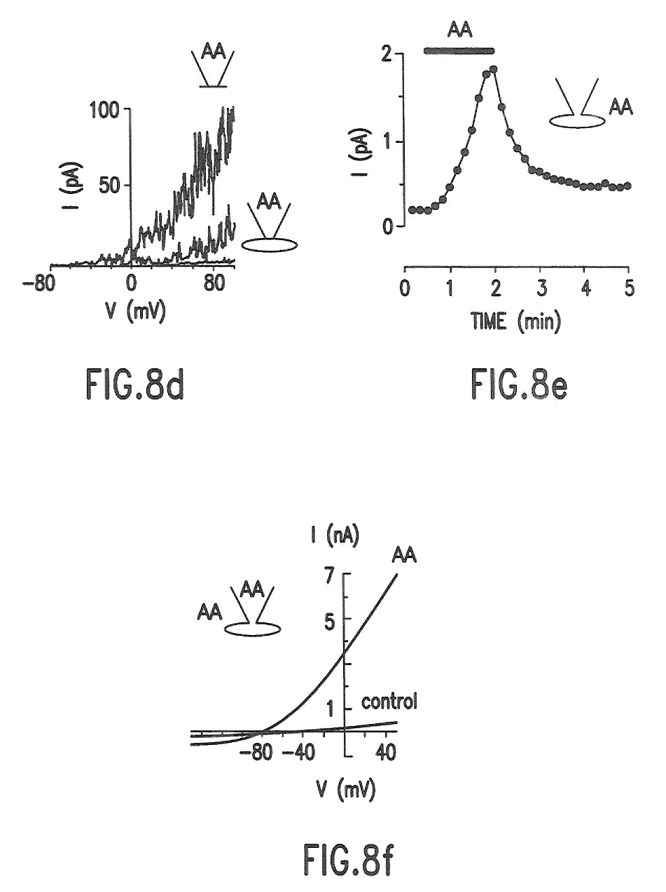

FIG. 8 shows the activation of TREK-1 by arachidonic acid in the transfected COS cells. In FIG. 8a: the activity of TREK-1 was recorded in the attached cell configuration. The patch was stimulated by a potential ramp lasting 800 msec every 5 seconds. The currents were measured at 80 mV. The applications of arachidonic acid (AA, 10 μM) are indicated by the horizontal bars. During the experiment, the patch was stimulated by tensions of 50 mmHg (indicated by the arrows). At 9 minutes, the patch was excised in the inside-out configuration. In FIG. 8b: current-potential relations corresponding to the experiment illustrated in FIG. 8a is shown. In FIG. 8c: activity of TREK-1 in the attached cell configuration with 10 μM AA in the pipette can be seen. The potential ramp lasted 800 msec and the currents were measured at 80 mV. In FIG. 8d: single-channel current-potential relations at the moment at which the pipette was placed on the membrane or after 20 minutes and 1 minute after the patch was excised in the inside-out configuration. In FIG. 8e: effect of AA (10 μM) on the TREK-1 current recorded in the intact cell is demonstrated. The current was measured at 80 mV. In FIG. 8f: AA had no effect on the TREK-1 current measured in the intact cell when it was in the pipette. The current was measured 30 minutes after the patch was broken (control tracing) by a potential ramp of 800 msec. The current was then measured after an application of AA of 1 minute in the external medium (AA tracing).

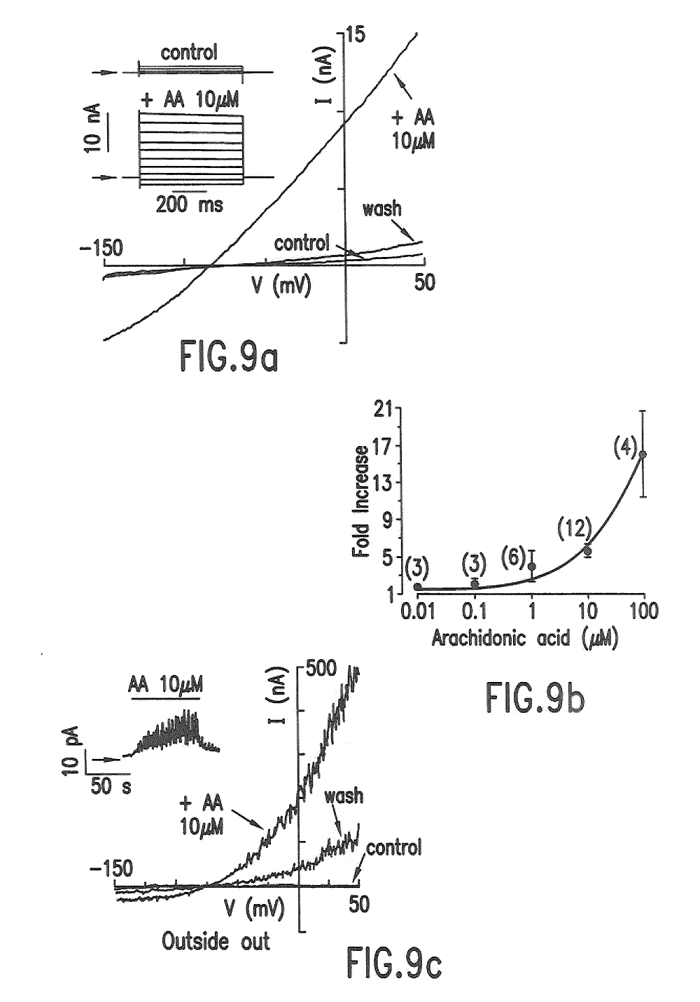

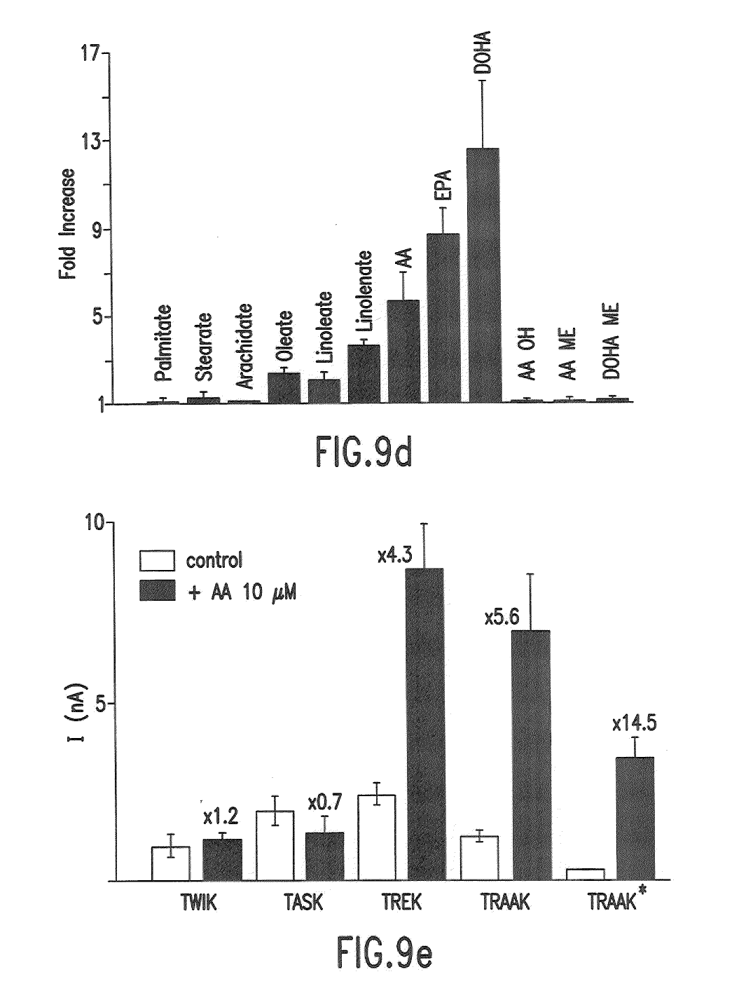

FIG. 9 shows the effect of arachidonic acid and other fatty acids on the TRAAK channel expressed in the transfected COS cells. In FIG. 9a: current-potential relations obtained from potential ramps of 5 W msec from −150 to +50 mV, after application of AA (10 μM) and after washing are shown. The inset shows the currents triggered by the potential jumps from −130 to +50 mV in increments of 20 mV. The maintenance potential was −80 mV. In FIG. 9b: dose-effect relation of the activation of TRAAK by AA is shown. In FIG. 9c: current-potential relations obtained as in FIG. 9a in the outside-out configuration are shown. The inset shows the effect of AA at 20 mV. In FIG. 9d: a histogram represents the coefficient of augmentation of the currents obtained after application of various fatty acids (10 μM). In FIG. 9e: the histogram shows the value of the currents, recorded in the intact cell configuration before and after application of AA on the cells temporarily transfected by TWIK-1, TASK, TREK-1 and TRAAK and on the cells-transfected in a stable manner by TRAAK. The coefficient of augmentation is indicated in each case.

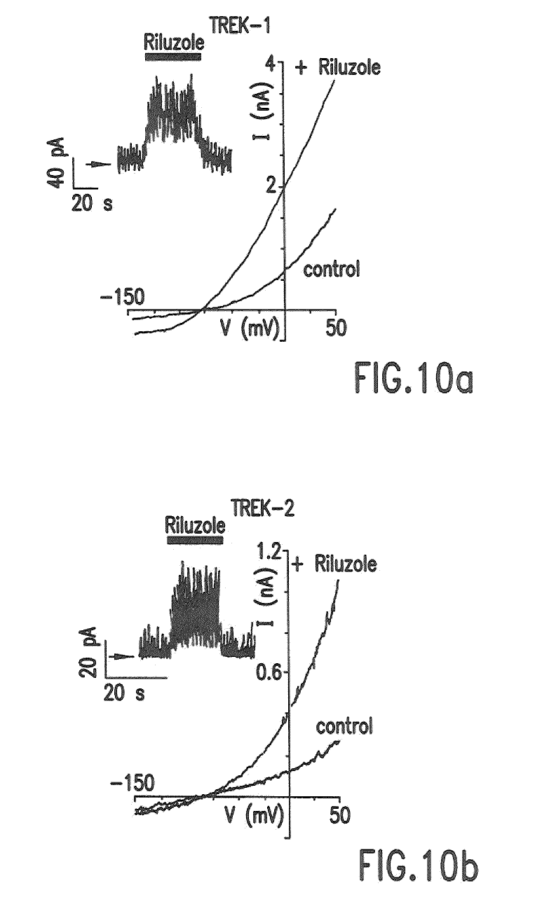

FIGS. 10a and b are graphs showing the effect of riluzole on the TREK-1 and TRAAK designated TREK-2 currents. The current-potential relations were obtained as in FIG. 9a above and after application of riluzole (100 μM) on the transfected COS cells. The inset shows the effects of riluzole on the currents recorded in the outside-out configuration.

### I. Cloning, Primary Structure and Tissue Distribution of TRAAK

The sequence of the TWIK-1 channel was used to detect homologous sequences in public DNA data libraries (Genbank and EMBL) employing the BLAST alignment program. It was thereby possible to identify a human TAG expressed sequence which was, used to screen a library of mouse brain cDNA. Multiple clones were isolated and characterized. The longest was sequenced. The following characteristics were determined:
The isolated cDNA-contained an open reading phase of 1197 nucleotides coding for a polypeptide of 398 residues. The nucleotide and protein sequences are shown in FIG. 1.
This protein contains four potential transmembranal segments and two P domains. It thus has the same general structure as the TWIK-1, TREK-1 and TASK channels. In addition, it exhibits sequence homologies with these channels: about 20-25% identity with TWIK-1 and TASK and about 38% identity with TREK-1. With the exception of the P domains which are present in all of the cloned potassium channels, it has no significant sequence homology with the channels of the Shaker and IRK type. It, therefore, belongs to the TWIK-1 family and its closest homologue is TREK-1. These relations can be seen in FIG. 2 at the level of the alignment of the protein sequences as well as in the dendrogram which was deduced from this alignment. TRAAK and TREK-1 thus form a structural subclass within the TWIK-1 family.

The sequences of various oligonucleotides were deduced from the sequence of TRAAK. These oligonucleotides enabled the use of RT-PCR to study the distribution of the transcript coding for TRAAK in adult mouse tissues. As shown in FIG. 3, TRAAK is exclusively expressed in the neural tissues brain, cerebellum, spinal cord and retina. This distribution is very different from that of its closest homologue which is the TREK-1 channel. This substance has an almost ubiquitous distribution and is present in the excitatory tissues as well as the nonexcitatory tissues.

### II. Functional Expression of TRAAK

For the functional study, the coding sequence of TRAAK was inserted in the vector pEXO and a complementary RNA (cRNA) was synthesized from this construction and injected in Xenopus oocytes. For expression in the COS cells, the TRAAK sequence was subcloned in an expression vector under the control of a eukaryote promoter and transfected into the cells. An absent non-inactivating current from the oocytes and the control cells was measured by the imposed voltage technique as represented in FIG. 4. The activation was instantaneous and could not be resolved because it was masked by the capacitive discharge of the current recorded at the beginning of the potential jump. The current-potential relation rectified in the outgoing direction when the external K⁺ concentration was equal to 2 mM. Incoming currents were observed when the external K⁺ concentration was increased. At all concentrations, the current-potential curves followed the Goldman-Hodgkin-Katz relation. This demonstrates that the TRAAK currents have no rectification other than that which is due to the dyssymmetrical concentrations of K⁺ on each side of the membrane and that TRAAK is a channel which is not potential-dependent. The TRAAK channel is selective for potassium. Reversal of the current potential follows the equilibrium potential of K⁺ and changing the concentration of K⁺ by 10 leads to a change in the potential inversion value conforming to the value predicted by Nernst's equation (48.7±0.7 mV times 10, n=4).

The properties of TRAAK, absence of activation and, inactivation kinetics as well as its opening at all membrane potentials, are the characteristics of the potassium channels known as leakage channels. As to be expected for channels of this type, their expression in oocytes is associated with a strong polarization. The resting potential of the membrane passes, from −43±2.4 mV (n=7) in the control oocytes to −88±1.4 mV (n=23) in the transfected oocytes, a value close to the equilibrium potential of potassium. TRAAK was also, expressed in the transfected COS-M6 cells. In this system as well, the TRAAK currents were instantaneous and were not inactivated. The recording of the patch in outside-out configuration indicated a unit conductance of TRAAK equal to 45.5±3.7 pS (n=10).

### III TREK-1 and TRAAK are Mechanosensitive Channels

It has, been established that the structural subclass formed by the TREK-1 and TRAAK K⁺ channels are associated with electrophysiological properties which are unique among the TWIK type K⁺ channels, The TREK-1 and TRAAK channels are, in fact, activated by a tension applied to the plasma, membrane. This tension is obtained either indirectly by changing the osmolarity of the external medium and thus the volume of the cell or more directly by applying a depression in the recording pipette. The following characteristics were demonstrated:
FIG. 5 demonstrates that the expression of the TREK-1 channel in the Xenopus oocytes, which were maintained in a hypotonic medium, induced instantaneous, non-inactivating currents. When the osmolarity of the external medium was increased by adding mannitol to it, a noteworthy decrease in the amplitude of the current of TREK-1 was seen which demonstrates a sensitivity of the channel to the cell volume. In contrast, the TASK channel is not affected by the osmolarity of the external medium.
FIG. 6 demonstrates that the TREK-1 channel is mechanosensitive. In the transfected COS cells and under resting conditions, the activity of TREK-1 was undetectable in the attached cell configuration whereas the activity of TASK was easily measurable under the same conditions. However, a depression applied to the membrane by means of the recording pipette triggered an opening of the TREK-1 channel. No such effect was seen with TASK. The activation of TREK-1 induced by the tension was also obtained in the inside-out configuration, i.e., when the patch was excised and the internal surface of the membrane was in contact with the external medium. In this configuration, the activity of the channel was also absent or very weak if tension was not applied to the membrane. The effect of the tension was gradual and an activation equal to half of the maximum value was detected for a depression equivalent to 23 mmHg. In addition, FIG. 6h shows that the activation induced by stretching is independent of the membrane potential.
FIG. 7 also shows that TRAAK is a channel activated by stretching. In the absence of depression or for low values, the TRAAK channel was inactive. For higher values, the channel was activated and a current was recorded. During the application of the depression, a decrease in the activity of the channel could be seen as was the case with TREK-1.

### IV. TREK-1 and TRAAK are Activated by Arachidonic Acid and Other Polyunsaturated Fatty Acids

Activation of the TREK-1 and TRAAK channels by mechanical stretching of the membrane is mimicked by the application of arachidonic acid and by the application of other polyunsaturated fatty acids but not by the application of saturated fatty acids. The following characteristics were demonstrated:
FIG. 8 demonstrates that TREK-1 is activated by arachidonic acid (AA). The application of AA on the control cells (CD8) had no effect. The activations obtained by stretching of the membrane and by application of AA are similar in amplitude but are not additive. The two, types of activation were suppressed in the attached cell configuration. When the recording pipette contained AA, excision of the patch in the inside-out configuration induced in a reproducible manner a noteworthy increase in the activity of TREK-1. Similarly, the amplitude of the activation induced by a depression applied in the recording pipette was greater when the patch was excised. Finally, it was seen that in the intact cell, internal AA did not activate TREK-1. When the cell was dialyzed for periods as long as 30 minutes, no channel activation from the internal AA took, place even though activation could be seen just a few seconds after the application of AA in the external medium. These, results indicate that AA activates TREK-1 solely when it is applied on the external surface of the membrane.

FIG. 9 demonstrates that the TRAAK channel is activated by AA in the same manner as TREK-1. The activation was reversible and dependent on the concentration applied. This activation was also seen in the outside-out configuration. Activation of TRAAK by A-A was not prevented when the AA perfusion contained a mixture of inhibitors of AA metabolism (norhydroguaiaretic acid for lipoxygenase; indomethacin for cyclooxygenase, clotrimazole for epoxygenase and ETYA which inhibits all of the metabolism pathways of AA, all at 10 mM). Under these conditions, the increase in the current induced by AA was 6.6±0.5 times (n=3) (at +50 mV). An increase of 1.7±0.4 times (n=3) in the background potassium current could be seen after administration of a cocktail of inhibitors in the absence of AA. This result demonstrates that the activation by AA does not require the transformation of the AA into eicosanoids.
FIG. 9 also demonstrates that fatty acids other than AA activate the channel. This activation is specific to the polyunsaturated cis fatty adds and was seen with oleic (C18Δ9), linoleic add (C18Δ912), linolenic (C18Δ9, 12, 15), eicosapentaenoic (EPA, C2OΔ5, 8, 11, 14, 17) and docosohexaenoic (DORA, C2OΔ4, 7, 10, 13, 16, 19) acids at a concentration of 10 mM. The saturated acids such as palmitic (C16), stearic (C18) and arachidic (C20) acids had no effect. The derivatives of AA and docosohexaenoic acid in which the carboxylic group is substituted by an alcohol group (AA-OH) or the methyl esters (AA-ME, DOHA-ME) are also inactive against TRAAK. The effect of AA on TRAAK can be seen on the cells that were transfected in a temporary manner as well as those transfected in a stable manner (three independent stable cell lines were tested).
Finally, FIG. 9 demonstrates that the effect of activation by AA is specific to TREK-1 and TRAAK. No effects of the same type were seen for the TWIK-1 and TASK channels.

In the oocytes, TRAAK was insensitive to the classic potassium channel blocking agents such as tetraethylammonium (TEA, 1 mM), 4-aminopyridine (4-AP, 1 mM) and quinine (100 mM). In contrast, Ba²⁺, (1 mM) blocked 56.7±4.6%, n=5, of the TRAAK current at +40 mV.

### V. The TREK-1 and TRAAK Channels are Activated by Riluzole, a Neuroprotective Agent

Riluzole is a neuroprotective agent used to prolong the survival of patients with amyotrophic lateral sclerosis. FIG. 10 demonstrates that this pharmacological agent is an opener of the TREK-1 and TRAAK channels. TREK-1 and TRAAK are the first ionic channels to exhibit activity stimulated by riluzole.

1. A purified protein comprising a mechanosensitive potassium channel activated by at least one polyunsaturated fatty acid and riluzole.

2. The purified protein of claim 1 wherein the polyunsaturated fatty acid is arachidonic acid.

3. The purified protein of claim 1 having the amino acid sequence set forth in SEQ ID NO: 4 or a functionally equivalent derivative thereof.

4. The purified protein of claim 1 corresponding substantially to the amino acid sequence set forth in SEQ. ID NO: 4 or a functionally equivalent derivative thereof.

5. Antibodies reactive with at least one purified protein of claim 4.

6. The antibodies of claim 5 wherein said antibodies are monoclonal.

7. A purified nucleic acid molecule, comprising a nucleic acid sequence encoding a protein of claim 4.

8. The nucleic acid molecule, of claim 7 wherein said molecule comprises nucleotides 484 to 1593 of the sequence set forth in SEQ ID NO: 3 or the complement thereof.

9. The nucleic acid molecule of claim 7 wherein said molecule comprises nucleotides 487 to 1593 of the sequence set forth in SEQ ID NO: 3 or the complement thereof.

10. A vector comprising at least one purified nucleic acid molecule of claim 7 operably linked to regulatory sequences.

11. A vector comprising at least one purified nucleic acid molecule of claim 8 operably linked to regulatory sequences.

12. A vector comprising at least one purified nucleic, acid molecule of claim 9 operably linked to regulatory sequences.

13. A method for producing the purified protein of claim 4 which comprises:
a) transferring the nucleic acid molecule of claim 7 into a cellular host;
b) culturing said host under suitable conditions to produce a protein comprising a potassium channel; and
c) isolating the protein of step (b).

14. A method for producing the purified protein of claim 4 which comprises:
a) transferring the vector of claim 10 into a cellular host;
b) culturing said host under suitable conditions to produce a protein comprising a potassium channel; and
c) isolating the protein of step (b).

15. A method for producing the purified protein of claim 4 which comprises:
a) transferring the nucleic acid molecule of claim 8 into a cellular host;
b) culturing said host under suitable conditions to produce a protein comprising a potassium channel; and
c) isolating the protein of step (b).

16. A method for producing the purified protein of claim 4 which comprises:
a) transferring the vector of claim 11 into a cellular host;
b) culturing said host under suitable conditions to produce a protein comprising a potassium channel; and
c) isolating the protein of step (b).

17. A method for producing the purified protein of claim 4 which comprises:
a) transferring the nucleic acid molecule of claim 9 into a cellular host;
b) culturing said host under suitable conditions to produce a protein comprising a potassium channel; and
c) isolating the protein of step (b).

18. A method for producing the purified protein of claim 4 which comprises:
a) transferring the vector of claim 12 into a cellular host;
b) culturing said host under suitable conditions, to produce a protein comprising a potassium channel; and
c) isolating the protein of step (b).

19. A method for expressing a potassium channel of claim 4 which comprises:
(a) transferring the purified nucleic acid molecule of claim 7 into a cellular host; and
(b) culturing said host under suitable conditions for expressing the potassium channel.

20. A method for expressing a potassium channel of claim 4 which comprises:
(a) transferring the vector of claim 10 into a cellular host; and
(b) culturing said host under suitable conditions for expressing the potassium channel.

21. A method for expressing a potassium channel of claim 4 which comprises:
(a) transferring the purified nucleic acid molecule of claim 8 into a cellular host; and
(b) culturing said host under suitable conditions for expressing the potassium channel.

22. A method for expressing a potassium channel of claim 4 which comprises:
(a) transferring the vector of claim 11 into a cellular host; and
(b) culturing said host under suitable conditions for expressing the potassium channel.

23. A method for expressing a potassium channel of claim 4 which comprises:
(a) transferring the purified nucleic acid molecule of claim 9 into a cellular host; and
(b) culturing said host under suitable conditions for expressing the potassium channel.

24. A method for expressing a potassium channel of claim 4 which comprises:
(a) transferring the vector of claim 12 into a cellular host; and
(b) culturing said host under suitable conditions for expressing the potassium channel.

25. A cellular host comprising a nucleic acid molecule of claim 7.

26. A cellular host comprising a nucleic acid molecule of claim 8.

27. A cellular host comprising a nucleic acid molecule of claim 9.

28. A cellular host comprising a vector of claim 10.

29. A cellular host comprising a vector of claim 11.

30. A cellular host comprising a vector of claim 12.

31. A method for screening substances capable of modulating the activity of the purified protein of claim 4 which comprises:
(a) reacting varying amounts of the substance to, be screened with a cellular host of claim 25; and
(b) measuring the effect of the substance to be screened, on a potassium channel expressed by the cellular host.

32. A method for screening substances capable of modulating the activity of the purified protein of claim 4 which comprises:
(a) reacting: varying amounts of the substance to be screened with a cellular host of claim 26; and
(b) measuring the effect of the substance to be screened on a potassium channel expressed by the cellular host.

33. A method for screening substances capable of modulating the activity of the purified protein of claim 4 which comprises:
(a) reacting varying amounts of the substance to be screened with a cellular host of claim 27; and
(b) measuring the effect, of the substance to be screened on a potassium channel expressed by the cellular host.

34. A method for screening substances capable of modulating the activity of the purified protein of claim 4 which comprises:
(a) reacting varying amounts of the substance to be screened with a cellular host of claim 28; and
(b) measuring the effect of the substance to be screened on a potassium channel expressed by the cellular host.

35. A method for screening substances capable, of modulating the activity of the purified, protein of claim 4 which comprises:
(a) reacting varying amounts of the substance to be screened with a cellular host of claim 29; and
(b) measuring the effect of the substance to be screened on a potassium channel expressed by the cellular host.

36. A method for screening substances capable of modulating the activity of the purified protein of claim 4 which comprises:
(a) reacting varying amounts of the substance to be screened with a cellular host of claim 30; and
(b) measuring the effect of the substance to be screened on a potassium channel expressed by the cellular host.

37. The process of claim 31 wherein said process screens substances capable of preventing or treating heart disease in mammals.

38. The process of claim 31 wherein said process screens substances capable of preventing or treating central nervous system disease in mammals.

39. A method for preventing or treating heart disease in mammals which comprises administering a therapeutically effective amount of a pharmaceutical composition comprising a therapeutically effective amount of a substance capable of modulating the activity of the purified protein of claim 4.

40. A method for preventing or treating central nervous system disease in mammals which comprises administering a therapeutically effective amount of a pharmaceutical composition comprising a therapeutically effective amount of a substance capable of modulating the activity of the purified protein of claim 4.

41. The method of claim 39 wherein said method is useful for preventing or treating cardiac pathologies and vascular diseases.

42. The method of claim 40 wherein said method is useful for preventing or treating neurodegenerative diseases.

43. A pharmaceutical composition comprising a therapeutically effective amount of at least one purified protein of claim 4 and a pharmaceutically acceptable carrier.

44. A pharmaceutical composition comprising a therapeutically effective amount of at least one antibody of claim 5 and a pharmaceutically acceptable carrier.

45. A pharmaceutical composition comprising a therapeutically effective amount of at least one antibody of claim 6 and a pharmaceutically acceptable carrier.

46. A pharmaceutical composition comprising a therapeutically effective amount of at least one purified nucleic acid molecule of claim 7 and a pharmaceutically acceptable carrier.

47. A pharmaceutical composition comprising a therapeutically effective amount of at least one purified nucleic acid molecule of claim 8 and a pharmaceutically acceptable carrier.

48. A pharmaceutical composition comprising a therapeutically effective amount of at least one purified nucleic acid molecule of claim 9 and a pharmaceutically acceptable carrier.

49. A pharmaceutical composition comprising a therapeutically effective amount of at least one vector of claim 10 and a pharmaceutically acceptable carrier.

50. A pharmaceutical composition comprising a therapeutically effective amount of at least one vector of claim 11 and a pharmaceutically acceptable carrier.

51. A pharmaceutical composition comprising a therapeutically effective amount of at least one vector of claim 12 and a pharmaceutically acceptable carrier.

52. An isolated peptide chain comprising the amino acid sequence shown in SEQ ID NO: 4.

53. An isolated peptide chain comprising amino acid residues 2 to 370 the amino acid sequence shown in SEQ ID NO:4.

54. An isolated nucleic acid comprising the nucleic acid sequence shown in SEQ ID NO: 3.

55. An isolated nucleic acid comprising nucleic acid residues 487 to 1593 of the nucleic acid sequence shown in SEQ ID NO: 3.

56. An isolated nucleic acid comprising nucleic acid, residues 484 to 1593 of the nucleic acid sequence shown in SEQ ID NO: 3.

57. A composition comprising an effective amount of at least one isolated peptide chain of claim 52 or 53.

58. A purified protein comprising a mechanosensitive potassium channel comprising four transmembranal segments and two P domains activated by at least one polyunsaturated fatty acid and riluzone wherein the purified protein is selected from the group consisting of a purified protein encoded by the nucleic acid sequence of SEQ ID NO: 3 and a purified protein comprising the amino acid sequence of SEQ ID NO: 4.

59. The purified protein of claim 58, wherein the polyunsaturated fatty acid is arachidonic acid.

60. A composition comprising an effective amount of at least one purified protein of claims 58 or 59 and an acceptable carrier.

61. The process of claim 32 wherein said process screens substances capable of preventing or treating heart disease in mammals.

62. The process of claim 33 wherein said process screens substances capable of preventing or treating heart disease in mammals.

63. The process of claim 34 wherein said process screens substances capable of preventing or treating heart disease in mammals.

64. The process of claim 35 wherein said process screens substances capable of preventing or treating heart disease in mammals.

65. The process of claim 36 wherein said process screens substances capable of preventing or treating heart disease in mammals.

66. The process of claim 32 wherein said process screens substances capable of preventing or treating central nervous system disease in mammals.

67. The process of claim 33 wherein said process screens substances capable of preventing or treating central nervous system disease in mammals.

68. The process of claim 34 wherein said process screens substances capable of preventing or treating central nervous system disease in mammals.

69. The process of claim 35 wherein said process screens substances capable of preventing or treating central nervous system disease in mammals.

70. The process of claim 36 wherein said process screens substances capable of preventing or treating central nervous system disease in mammals.


SEQUENCE LISTING (6 sequences)

SEQ ID NO: 1 - DNA, 1757 bp, Unknown Sequence
  Description of Unknown Sequence DNA encoding TRAAK
ccacgcgtccgcggacgcgtgggtcgcccacgcgtccggtggcggctgtcctgagccccgggccagctgatgtccaggttagggcagcgttggggccccaatcccagcctggaaggttggacttcacgtcgacccttctctgagtcttctgccactcactggcctggacaagacagcattggggagcccagaggctgcaggtgcagtgaccactgctccccaggagctccctgctccttcttcccaggcaggaagtggagctggacctgcctctggaaggaccatgcgcagcaccacactcctggctctgctggcactggtgctgctttacttggtatctggggctctagtgttccaggctctggagcagcctcacgagcagcaggctcagaagaaaatggatcatggccgagaccagtttctgagggaccatccctgtgtgagccagaagagcctggaggatttcatcaagctcctggttgaagccctgggagggggcgcaaacccagaaaccagctggaccaatagcagcaaccactcatcagcttggaacctgggcagcgccttctttttctcggggaccatcatcactaccatcggctatggcaatatagtcttacacacagatgccgggcgtctcttttgtatcttctatgcactggtggggatcccactgttcgggatgctgctggcgggagtcggggaccggctgggctcctctctgcgccggggcatcggccacatcgaagcaatcttcttgaagtggcatgtgccaccggggctggtgagaagtctgtccgcagtgctcttcctgctgatcggctgcctgctctttgtcctcactcctaccttcgtgttctcctacatggagagctggagcaagttagaagccatctactttgttatagtgactctcaccactgtaggctttggcgattatgtacccggcgatggcaccgggcagaactctccagcctaccagccgctggtgtggttctggatcttgtttggcctagcctacttcgcctcagtgctcaccaccatcggcaactggttgcgagcagtgtcccgccgaactcgggcagagatgggtggcctaacggcacaggctgctagctggaccggcacagtgacagcgcgagtgacccagcgaactgggcccagcgccccgccgccagagaaggagcaaccactcctgccctcctctttgccggcaccgcctgctgttgttgagccagccggcaggcccggctcccctgcacccgcagagaaggttgagactccgtccccgcccacggcctcagctctggattaccccagtgagaatctggccttcatcgacgagtcctcagacacgcagagtgagcgtggctgtgccctgcctcgggctcctcggggtcgccgccgacccaacccatccaaaaagccttccagaccccggggtcctgggcgactccgagacaaggccgtgccggtgtaggggcaggatctctggacccggatcccacgccagggctttcgctcttgctgatgctcaggcatgcttggcttatttgaccaaagagccgtccctcttttgttccacgtggttgcaaccctgacaggagtccagtggttgccaaatgccaccgctcttccctggctggttcttcacatccaatcatttccaaagcccaccatccaaggctttctgcctcgctcccctgccggttttgaccctcacacctcacaactgtgcctcaaaacctgcaccaata

SEQ ID NO: 2 - protein, 398 aa, Unknown Sequence
  Description of Unknown Sequence TRAAK
MRSTTLLALLALVLLYLVSGALVFQALEQPHEQQAQKKMDHGRDQFLRDHPCVSQKSLEDFIKLLVEALGGGANPETSWTNSSNHSSAWNLGSAFFFSGTIITTIGYGNIVLHTDAGRLFCIFYALVGIPLFGMLLAGVGDRLGSSLRRGIGHIEAIFLKWHVPPGLVRSLSAVLFLLIGCLLFVLTPTFVFSYMESWSKLEAIYFVIVTLTTVGFGDYVPGDGTGQNSPAYQPLVWFWILFGLAYFASVLTTIGNWLRAVSRRTRAEMGGLTAQAASWTGTVTARVTQRTGPSAPPPEKEQPLLPSSLPAPPAVVEPAGRPGSPAPAEKVETPSPPTASALDYPSENLAFIDESSDTQSERGCALPRAPRGRRRPNPSKKPSRPRGPGRLRDKAVPV

SEQ ID NO: 3 - DNA, 1993 bp, Unknown Organism
  Description of Unknown Organism DNA encoding TREK
agagcggcgaggcgaggggagagtggtgctacgggccaggcgggccaccccgggccacacccccaccttgcgggcgcccggcggggctcgagccaggcggggcgcctcacaaagacatgcgaagaggggctgcagtgatcaccccctcgctgagccccggggcagagcccagccgccggccgagcgcacggagccacgggccgagcgcacccagggcccgcgcgggaccccaggcggccacgcaatcggggtgacccatcgcgcgcgggggcgtcgtcgtccgatcccaacttggcctcggcctcgccctctgcccagcctgccaccgctggtgtcctctccttccggcgatttcgtttcttctcacgctcccccctctatacccctcccgcctccagccccgctctccccaccttgtaaaacaaagccggggaaaatgcctacccgtgcagctcggagcgcgcagcccgtcttggaataaggatggcggcccctgacttgctggatcccaagtctgctgctcagaactccaaaccgaggctctcattctcttcaaaacccaccgtgcttgcttcccgggtggagagtgactcggccattaatgttatgaaatggaagacagtctccacgattttcctggtggtcgtcctctacctgatcatcggagccgcggtgttcaaggcattggagcagcctcaggagatttcccagaggaccaccattgtgatccagaagcagaccttcatagcccagcatgcctgcgtcaactccaccgagctggacgaactcatccagcaaatagtggcagcaataaacgcagggattatccccttaggaaacagctccaatcaagttagtcactgggacctcggaagctctttcttctttgctggtactgttatcacaaccataggatttggaaacatctccccacgaactgaaggtggaaaaatattctgcatcatctatgccttgctgggaattcccctctttggctttctactggctggggttggtgatcagctaggaactatatttggaaaaggaattgccaaagtggaagacacatttattaagtggaatgttagtcagacgaagattcgtatcatctccaccatcatcttcatcctgtttggctgtgtcctctttgtggctctccctgcggtcatattcaagcacatagaaggctggagcgccctggacgctatctattttgtggttatcactctgacgaccattggatttggagactacgtggcaggtggatcagacattgaatatctggacttctacaagcctgtggtgtggttctggatcctcgttgggctggcctactttgcagctgttctgagcatgattggggactggctacgggtgatctctaagaagacgaaggaagaggtgggagagttcagagcgcatgccgctgagtggacagccaatgtcacggccgagttcaaggaaacgaggaggcggctgagcgtggagatctacgacaagttccagcgtgccacatccgtgaagcggaagctctccgcagagctggcgggcaaccacaaccaggaactgactccgtgtatgaggacctgtctgtgaaccacctgaccagcgagagggaagtcctgcctcccttgctgaaggctgagagcatctatctgaacggtctgacaccacactgtgctggtgaggacatagctgtcattgagaacatgaagtagccctctcttggaagagtctgaggtggagccatagggaagggcttctctaggctctttgtgactgttgccggtagcatttaaacattgtgcatggtgacctcaaagggaaagcaaatagaaaacacccatctggtcaccttacatccagggagggtgttgtcccgaggcggcactctgaggatgccgtgtgctgtccgctgagtgctgagtgatggacaggcagtgtctgatgccttttgtgcccagactgtttcccctccccctctctcctaacg

SEQ ID NO: 4 - protein, 370 aa, Unknown Organism
  Description of Unknown Organism TREK
MAAPDLLDPKSAAQNSKPRLSFSSKPTVLASRVESDSAINVMKWKTVSTIFLVVVLYLIIGAAVFKALEQPQEISQRTTIVIQKQTFIAQHACVNSTELDELIQQIVAAINAGIIPLGNSSNQVSHWDLGSSFFFAGTVITTIGFGNISPRTEGGKIFCIIYALLGIPLFGFLLAGVGDQLGTIFGKGIAKVEDTFIKWNVSQTKIRIISTIIFILFGCVLFVALPAVIFKHIEGWSALDAIYFVVITLTTIGFGDYVAGGSDIEYLDFYKPVVWFWILVGLAYFAAVLSMIGDWLRVISKKTKEEVGEFRAHAAEWTANVTAEFKETRRRLSVEIYDKFQRATSVKRKLSAELAGNHNQELTPCMRTCL

SEQ ID NO: 5 - protein, 336 aa, Unknown Sequence
  Description of Unknown Sequence TWIK
MLQSLAGSSCVRLVERHRSAWCFGFLVLGYLLYLVFGAVVFSSVELPYEDLLRQELRKLKRRFLEEHECLSEQQLEQFLGRVLEASNYGVSVLSNASGNWNWDFTSALFFASTVLSTTGYGHTVPLSDGGKAFCIIYSVIGIPFTLLFLTAVVQRITVHVTRRPVLYFHIRWGFSKQVVAIVHAVLLGFVTVSCFFFIPAAVFSVLEDDWNFLESFYFCFISLSTIGLGDYVPGEGYNQKFRELYKIGITCYLLLGLIAMLVVLETFCELHELKKFRKMFYVKKDKDEDQVHIIEHDQLSFSSITDQAAGMKEDQKQNEPFVATQSSACVDGPANH

SEQ ID NO: 6 - protein, 394 aa, Unknown Sequence
  Description of Unknown Sequence TASK
MKRQNVRTLALIVCTFTYLLVGAAVFDALESEPELIERQRLELRQQELRARYNLSQGGYEELERVVLRLKPHKAGVQWRFAGSFYFAITVITTIGYGHAAPSTDGGKVFCMFYALLGIPLTLVMFQSLGERINTLVRYLLHRAKKGLGMRRADVSMANMVLIGFFSCISTLCIGAAAFSHYEHWTFFQAYYYCFITLTTIGFGDYVALQKDQALQTQPQYVAFSFVYILTGLTVIGAFLNLVVLRFMTMNAEDEKRDAEHRALLTRNGQAGGGGGGGSAHTTDTASSTAAAGGGGFRNVYAEVLHFQSMCSCLWYKSREKLQYSIPMIIPRDLSTSDTCVEQSHSSPGGGGRYSDTPSRRCLCSGAPRSAISSVSTGLHSLSTFRGLMKRRSSV